(19) United States
(12) Patent Application Publication
(10) Pub. No.: US 2007/0286812 A1
(43) Pub. Date: Dec. 13, 2007

(54) NASAL FORMULATION

(76) Inventor: Camille TOUTOUNGHI, Montreal (CA)

Correspondence Address: BERESKIN AND PARR, 40 KING STREET WEST, BOX 401, TORONTO, ON, M5H 3Y2, US

(21) Appl. No.: 11/423,246

(22) Filed: Jun. 9, 2006

(51) Int. Cl. A61K 35/74; A61K 36/8962; A61K 33/38; A61K 36/53; A61K 31/7048; A61K 31/7034; A61K 9/12; A61K 31/496; A61K 31/4196; A61K 31/4178

(52) U.S. Cl. 424/45; 424/93.45; 424/618; 424/745; 424/754; 514/254.07; 514/383; 514/397; 514/28; 514/35; 514/22

(57) ABSTRACT

The present invention relates to a novel nasal formulation for nasal hygiene and to improve nasal airflow of people suffering from nasal congestion.

### BACKGROUND OF THE INVENTION

(a) Field of the Invention

The present invention relates to a novel nasal formulation for nasal hygiene and to improve nasal airflow of people suffering from nasal congestion.

(b) Description of Prior Art

Many people suffer from nasal congestion worldwide and successful treatment remains lacking.

Nasal congestion occurs when the membranes lining the nose become swollen and inflamed, along with dilation of the blood vessels. Congestion can be caused by many factors, including colds, allergies, sinus infections, and the flu. Overuse of some nasal sprays or drops can also lead to congestion as a rebound effect.

A stuffy nose is usually caused by a virus and typically resolves spontaneously within a week to ten days.

Newborn infants must breathe through the nose. Nasal congestion in an infant in the first few months of life can interfere with nursing and cause life-threatening breathing problems. Nasal congestion in older children and adolescents is often just an annoyance but can cause other difficulties.

Nasal congestion can interfere with the ears, hearing, and speech development. Significant congestion may interfere with sleep, cause snoring, and can be associated with episodes of not breathing during sleep (sleep apnea). In children, nasal congestion from enlarged adenoids has caused chronic sleep apnea with insufficient oxygen levels and right-sided heart failure. The problem usually resolves after surgery to remove the adenoids and tonsils.

Common Causes:

Common cold or the flu—often accompanied by a clear, watery discharge plus fever, cough, headache, joint aches, muscle aches, or sore throat.

Hay fever—often accompanied by a clear, watery discharge, itching eyes, and sneezing.

Sinus infection—often accompanied by thick, cloudy, yellow-green nasal discharge, and pain or tenderness around eyes and cheekbones that worsens when bending the head forward.

To relieve a runny or stuffy nose, the following over-the-counter medicines may help:

Decongestants—these may help relieve congestion by shrinking the blood vessels in the lining of the nose. They only help with stuffiness, not a runny nose or other symptoms. Decongestant nasal sprays and drops should not be used for more than 3 days, because then they can make the congestion worse.

Antihistamines—these may reduce the amount of mucus. However, some antihistamines make people drowsy.

These medicines can make breathing more comfortable, but do not treat the underlying condition. Many over-the-counter allergy and cold medicines contain multiple ingredients.

Medicines are not the only way to relieve a stuffy or runny nose. Try these steps to thin the mucus, which can help to make breathing easier and get the nasal secretions back to normal:

Use gentle saline nasal sprays.

Increase the humidity in the air with a vaporizer or humidifier.

Drink extra fluids. Hot tea, broth, or chicken soup may be especially helpful.

Pharmacies and supermarkets sell adhesive strips that can be placed on the nose for nasal congested. These help widen the nasal passages, making breathing easier.

To date there exist no known treatment for nasal congestion not associated with rhinoviral infection or bacterial sinusitis.

U.S. Pat. No. 6,207,703 in the name of Ponikau discloses a method for treating and preventing non-invasive fungus-induced mucositis, rhinosinusitis, chronic otitis media, chronic colitis, Crohn's disease and chronic asthma without any mention of effects on nasal airflow or congestion.

It would be highly desirable to be provided with a nasal formulation and a method for improving nasal airflow of a human suffering from nasal congestion not associated with rhinoviral infection, allergic rhinitis, or bacterial sinusitis, which method would overcome the drawbacks of the previous methods.

### SUMMARY OF THE INVENTION

In accordance with the present invention there is provided a nasal formulation and a method for improving nasal airflow of a human suffering from nasal congestion not associated with rhinoviral infection, allergic rhinitis, or bacterial sinusitis, which method would overcome the drawbacks of the previous methods.

In accordance with an embodiment of the present invention there is provided a method for improving nasal airflow of a human suffering from nasal congestion, said method comprising administering nasally to said human a decongestant effective amount of an antifungal agent.

In accordance with an embodiment of the present invention there is provided a nasal formulation for improving nasal airflow of a human suffering from nasal congestion, comprising a decongestant effective amount of an antifungal agent or a combination of antifungal agents in association with a nasal inert carrier.

In accordance with an embodiment of the present invention there is provided a nasal formulation for improving nasal airflow of a human suffering from nasal congestion, comprising a synergistic amount of a pharmaceutical antifungal agent or a combination thereof in combination with a compound chosen from a natural antifungal agent, a vasoconstrictor, a glucocorticosteroid, an antihistamine, an anticholinergic, a mucolytic, and a combination thereof, in association with a nasal inert carrier.

In accordance with the present invention, the antifungal agent is chosen from a pharmaceutical antifungal agent and a natural antifungal agent.

As an example, such a pharmaceutical antifungal agent may be chosen from terbinafine, butenafine, naftifine, butoconazole, clotrimazole, econazole, fluconazole, itraconazole, ketoconazole, miconazole, oxiconazole, sulconazole, sertaconazole, terconazole, saperconazole, tinidazole, tioconazole, voriconazole, nyastatin, micafungin, griseofulvin, haloprogin, mafenide, mycafungin, caspofungin, flucytosine, natamycin, undecylenic acid, caprylic acid, propionic acid, ciclopyrox olamine, amphotericin B, and tolnaftate.

As an example, such a natural antifungal agent may chosen from oregano oil, berberine, garlic oil, allicin, alliin, alliinase, cellulase, S-allylcysteine, colloidal silver, tannin, Lactobacillus acidophilus, Lactobacillus rhamnosus, Lactobacillus bulgaricus, Lactobacillus salivarius, Lactobacillus plantarum, Lactobacillus casei, and Lactobacillus sporogenes, Bifidobacteria bifidum, Bifidobacteria longum, and Bifidobacteria infantis.

In accordance with the present invention, the formulation may be in a solid, liquid or aerosol form, such as, without limitation, a spray, aerosol, powder, mist, nebulae, ointment, cream, gel, paste, salve, solution, suspension, tincture, patch, and atomized vapor.

For the purpose of the present invention the following terms are defined below.

The term “decongestant effective amount” is intended to mean the amount required to have a decongestant effect by improving nasal airflow of a human suffering from nasal congestion.

The term “antifungal agent” is intended to mean any known pharmaceutical antifungal agents and any known natural antifungal agents.

The term “pharmaceutical antifungal agent” is intended to mean any synthetic and/or pharmaceutical antifungal agents, which include without limitation, those chosen from terbinafine, butenafine, naftifine, butoconazole, clotrimazole, econazole, fluconazole, itraconazole, ketoconazole, miconazole, oxiconazole, sulconazole, sertaconazole, terconazole, saperconazole, tinidazole, tioconazole, voriconazole, nyastatin, micafungin, griseofulvin, haloprogin, mafenide, mycafungin, caspofungin, flucytosine, natamycin, undecylenic acid, caprylic acid, propionic acid, ciclopyrox olamine, amphotericin B, and tolnaftate.

The term “natural antifungal agent” is intended to mean any substances referred to as having a natural origin, which include without limitation, those chosen from oregano oil, berberine, garlic oil, allicin, alliin, alliinase, cellulase, s-allylcysteine, colloidal silver, tannin, Lactobacilli such as Lactobacillus acidophilus, Lactobacillus rhamnosus, Lactobacillus bulgaricus, Lactobacillus salivarius, Lactobacillus plantarum, Lactobacillus casei, and Lactobacillus sporogenes, and Bifidobacteria such as Bifidobacteria bifidum, Bifidobacteria longum, and Bifidobacteria infantis

The term “vasoconstrictor” is intended to mean any known substances having a vasoconstrictor effect, which include without limitation those chosen from phenylephrine hydrochloride, pseudoephedrine hydrochloride, oxymetazoline hydrochloride, xylometazoline hydrochloride, phenylpropanolamine, propylhexedrine and epinephrine.

The term “glucocorticosteroid” is intended to mean any known substances having a glucocorticosteroid effect, which include without limitation those chosen from triamcinolone, budesonide, beclomethasone, flunisolide, fluticasone, mometasone, dexamethasone, predisone, prednisolone, and hydrocortisone.

The term “antihistamine” is intended to mean any known substances having an antihistamine effect, which include without limitation those chosen from cetirizine, cromolyn, diphenhydramine, brompheniramine, chlorpheniramine and clemastine.

The term “anticholinergic” is intended to mean any known substances having an anticholinergic effect, which include without limitation ipratropium.

The term “mucolytic” is intended to mean any known substances having an mucolytic effect, which include without limitation those chosen from bromhexine, N-acetyl cysteine, amylase, protease, glucoamylase, betaglucanase, alpha-galactosidase, cellulase, xylanase, seaprose, pectinase, phytase, hemmicellulase and invertase.

The term “synergistic amount” is intended to mean the amount required to have a synergistic effect between two or more substances of a combination to improve nasal airflow of a human suffering from nasal congestion, wherein the effect of the substances in the combination is greater than the sum of the substances separately.

### DETAILED DESCRIPTION OF THE INVENTION

In accordance with the present invention, there is provided novel nasal formulations for improving nasal airflow of a human suffering from nasal congestion not associated with rhinoviral infection, allergic rhinitis or bacterial sinusitis.

The formulation of the present invention may include any of the following solvent base or inert carrier, which include, without limitation, water, purified water, saline, liposome, mineral oil, gel, ointment, alcohol and acetic acid.

The formulation of the present invention may include any of the following inactive ingredients, which include, without limitation, oil of eucalyptus (0.001% to 10%), oil of cloves (0.001% to 10%), oil of lavender (0.001% to 10%), oil of menthol (0.001% to 10%), oil of cinnamon (0.001% to 10%), xylitol (0.001% to 10%), and other aromatic substances.

In accordance with one embodiment of the present invention there is provided a nasal formulation containing between 0.001% to 25% of a pharmaceutical antifungal agent or combination thereof.

In accordance with another embodiment of the present invention there is provided a nasal formulation containing from 0.001% to 25% of a pharmaceutical antifungal agent in a combination with 0.001% to 25% of a different pharmaceutical antifungal agent.

In accordance with another embodiment of the present invention there is provided a nasal formulation containing from 0.001% to 25% of a pharmaceutical antifungal agent or combination thereof in a synergistic combination with 0.001% to 25% of a natural antifungal agent or combination thereof.

In accordance with another embodiment of the present invention there is provided a nasal formulation containing from 0.001% to 25% of a pharmaceutical antifungal agent or combination thereof in a synergistic combination with 0.001% to 25% of a glucocorticosteroid or combination thereof.

In accordance with another embodiment of the present invention there is provided a nasal formulation containing from 0.001% to 25% of a pharmaceutical antifungal agent or combination thereof in a synergistic combination with 0.001% to 25% of a natural antifungal agent or combination thereof and 0.001% to 25% of a glucocorticosteroid or combination thereof.

In accordance with another embodiment of the present invention there is provided a nasal formulation containing from 0.001% to 25% of a pharmaceutical antifungal agent or combination thereof in a synergistic combination with 0.001% to 25% of a vasoconstrictor or combination thereof.

In accordance with another embodiment of the present invention there is provided a nasal formulation containing from 0.001% to 25% of a pharmaceutical antifungal agent or combination thereof in a synergistic combination with 0.001% to 25% of a natural antifungal agent or combination thereof and 0.001% to 25% of one vasoconstrictor or combination thereof.

In accordance with another embodiment of the present invention there is provided a nasal formulation containing from 0.001% to 25% of a pharmaceutical antifungal agent or combination thereof in a synergistic combination with 0.001% to 25% of an antihistamine or combination thereof.

In accordance with another embodiment of the present invention there is provided a nasal formulation containing from 0.001% to 25% of a pharmaceutical antifungal agent or combination thereof in a synergistic combination with 0.001% to 25% of a natural antifungal agent or combination thereof and 0.001% to 25% of an antihistamine or combination thereof.

In accordance with another embodiment of the present invention there is provided a nasal formulation containing from 0.001% to 25% of a pharmaceutical antifungal agent or combination thereof in a synergistic combination with 0.001% to 25% of an anticholinergic or combination thereof.

In accordance with another embodiment of the present invention there is provided a nasal formulation containing from 0.001% to 25% of a pharmaceutical antifungal agent or combination thereof in a synergistic combination with 0.001% to 25% of a natural antifungal agent or combination thereof and 0.001% to 25% of an anticholinergic or combination thereof.

In accordance with another embodiment of the present invention there is provided a nasal formulation containing from 0.001% to 25% of a pharmaceutical antifungal agent or combination thereof in a synergistic combination with 0.001% to 25% of a vasoconstrictor or combination thereof and 0.001% to 25% of an antihistamine or combination thereof.

In accordance with another embodiment of the present invention there is provided a nasal formulation containing from 0.001% to 25% of a pharmaceutical antifungal agent or combination thereof in a synergistic combination with 0.001% to 25% of a natural antifungal agent or combination thereof 0.001% to 25% of one vasoconstrictor or combination thereof and 0.001% to 25% of an antihistamine or combination thereof.

In accordance with another embodiment of the present invention, as an example, antifungal agents are Butenafine, Terconazole, Terbinafine, Miconazole, Ketoconazole, Econazole, and Clotrimazole, Butoconazole.

In accordance with another embodiment of the present invention, examples of a formulation are as follows:

Terbinafine 0.1% solution in saline;

Terbinafine 0.1% solution in saline+Oil of Oregano 1%;

Butenafine 0.1% solution in saline;

Butenafine 0.1% solution in saline+Oil of Oregano 1%;

Terconazole 0.04% solution in saline;

Terconazole 0.04% solution in saline+Oil of Oregano 1%;

Miconazole 0.2% solution in saline;

Miconazole 0.2% solution in saline+Oil of Oregano 1%;

Miconazole 0.4% solution in saline;

Miconazole 0.4% solution in saline+Oil of Oregano 1%;

Ketoconazole 0.2% solution in saline;

Ketoconazole 0.2% solution in saline+Oil of Oregano 1%;

Econazole 0.1% solution in saline;

Econazole 0.1% solution in saline+Oil of Oregano 1%;

Clotrimazole 0.2% solution in saline;

Clotrimazole 0.2% solution in saline+Oil of Oregano 1%;

Butoconazole 0.2% solution in saline; and

Butoconazole 0.2% solution in saline+Oil of Oregano 1%.

In accordance with another embodiment of the present invention, the nasal formulation may be administered under the following regimen, twice a day for 7 to 10 days.

The present invention will be more readily understood by referring to the following examples which are given to illustrate the invention rather than to limit its scope.

### EXAMPLE 1

### Nasal Miconazole/Oregano Oil Formulation

A nasal miconazole/oregano oil formulation is prepared by dissolving 10 g miconazole 4% cream (obtained from Johnson & Johnson) and 1 ml oregano oil (obtained from North American Herb & Spice) in 89 ml of a saline solution (sodium chloride 0.65% obtained from Albertsons Inc). The resulting formulation has 0.4% miconazole and 1% oregano oil.

### EXAMPLE 2

### Treating Nasal Congestion

Two congested patients (age and sex of patient to be completed) were nasally administered with the formulation of Example 1 disposed in an atomizer. The formulation was used at a dose of one spray per nostril, three times a day for 10 days. Each patient reported a marked improvement of their nasal airflow and ease in breathing after only 3 days of use.

### EXAMPLE 3

### Nasal Ketoconazole Formulation

A nasal ketoconazole formulation is prepared by dissolving 10 g ketoconazole 2% cream (obtained from Janssen-Cilag) in 90 ml of water. The resulting formulation has 0.2% ketoconazole.

### EXAMPLE 4

### Treating Nasal Congestion

Three congested patients (age and sex of patient to be completed) were nasally administered with the formulation of Example 3 disposed in an atomizer. The formulation was used at a dose of one spray per nostril, three times a day for 14 days. Each patient reported a marked improvement of their nasal airflow and ease in breathing after only 5 days of use.

### EXAMPLE 5

### Nasal Terbinafine Formulation

A nasal terbinafine formulation is prepared by dissolving 10 g terbinafine 1% cream (obtained from Novartis) in 90 ml of a saline solution (sodium chloride 0.65% obtained from Albertsons Inc). The resulting formulation has 0.1% terbinafine.

### EXAMPLE 6

### Treating Nasal Congestion

One congested patient (age and sex of patient to be completed) was nasally administered with the formulation of Example 5 disposed in an atomizer. The formulation was used at a dose of one spray per nostril, three times a day for 14 days. The patient reported a marked improvement of his nasal airflow and ease in breathing after only 4 days of use.

### EXAMPLE 7

### Nasal Terbinafine/Oregano Oil Formulation

A nasal terbinafine/oregano oil formulation is prepared by dissolving 10 g terbinafine 1% cream (obtained from Novartis) and 1 ml oregano oil (obtained from North American Herb & Spice) in 89 ml of a saline solution (sodium chloride 0.65% obtained from Albertsons Inc). The resulting formulation has 0.1% terbinafine and 1% oregano oil.

### EXAMPLE 8

### Treating Nasal Congestion

Two congested patients (age and sex of patients to be completed) were nasally administered with the formulation of Example 7 disposed in an atomizer. The formulation was used at a dose of one spray per nostril, three times a day for 7 days. The patients reported a marked improvement of nasal airflow and ease in breathing after only 2 days of use.

### EXAMPLE 9

### Nasal Butenafine/Oregano Oil Formulation

A nasal butenafine/oregano oil formulation is prepared by dissolving 10 g butenafine 1% cream (obtained from Schering-Plough) and 1 ml oregano oil (obtained from North American Herb & Spice) in 89 ml of a saline solution (sodium chloride 0.65% obtained from Albertsons Inc). The resulting formulation has 0.1% butenafine and 1% oregano oil.

### EXAMPLE 10

### Treating Nasal Congestion

Two congested patients (age and sex of patients to be completed) were nasally administered with the formulation of Example 9 disposed in an atomizer. The formulation was used at a dose of one spray per nostril, three times a day for 14 days. The patients reported a marked improvement of nasal airflow and ease in breathing after only 2 days of use.

### EXAMPLE 11

### Nasal Econazole/Oregano Oil Formulation

A nasal econazole/oregano oil formulation is prepared by dissolving 10 g econazole 1% cream (obtained from Bristol-Myers Squibb) and 1 ml oregano oil (obtained from North American Herb & Spice) in 89 ml of a saline solution (sodium chloride 0.65% obtained from Albertsons Inc). The resulting formulation has 0.1% econazole and 1% oregano oil.

### EXAMPLE 12

### Treating Nasal Congestion

Two congested patients (age and sex of patients to be completed) were nasally administered with the formulation of Example 11 disposed in an atomizer. The formulation was used at a dose of one spray per nostril, three times a day for 14 days. The patients reported a marked improvement of nasal airflow and ease in breathing after only 4 days of use.

### EXAMPLE 13

### Nasal Clotrimazole/Oregano Oil Formulation

A nasal clotrimazole/oregano oil formulation is prepared by dissolving 10 g clotrimazole 2% cream (obtained from Bayer) and 1 ml oregano oil (obtained from North American Herb & Spice) in 89 ml of a saline solution (sodium chloride 0.65% obtained from Albertsons Inc). The resulting formulation has 0.2% clotrimazole and 1% oregano oil.

### EXAMPLE 12

### Treating Nasal Congestion

One congested patients (age and sex of patient to be completed) was nasally administered with the formulation of Example 13 disposed in an atomizer. The formulation was used at a dose of one spray per nostril, three times a day for 10 days. The patient reported a marked improvement of nasal airflow and ease in breathing after only 3 days of use.

While the invention has been described in connection with specific embodiments thereof, it will be understood that it is capable of further modifications and this application is intended to cover any variations, uses, or adaptations of the invention following, in general, the principles of the invention and including such departures from the present disclosure as come within known or customary practice within the art to which the invention pertains and as may be applied to the essential features hereinbefore set forth, and as follows in the scope of the appended claims.

1. A nasal formulation for improving nasal airflow of a human suffering from nasal congestion, comprising a decongestant effective amount of an antifungal agent or a combination of antifungal agents in association with a nasal inert carrier.

2. The nasal formulation of claim 1, wherein said antifungal agent is chosen from a pharmaceutical antifungal agent and a natural antifungal agent.

3. The nasal formulation of claim 2, wherein said pharmaceutical antifungal agent is chosen from terbinafine, butenafine, naftifine, butoconazole, clotrimazole, econazole, fluconazole, itraconazole, ketoconazole, miconazole, oxiconazole, sulconazole, sertaconazole, terconazole, saperconazole, tinidazole, tioconazole, voriconazole, nyastatin, micafungin, griseofulvin, haloprogin, mafenide, mycafungin, caspofungin, flucytosine, natamycin, undecylenic acid, caprylic acid, propionic acid, ciclopyrox olamine, amphotericin B, and tolnaftate.

4. The nasal formulation of claim 3, wherein said formulation is in a solid, liquid or aerosol form.

5. The nasal formulation of claim 3, wherein said formulation is in a form chosen from a spray, aerosol, powder, mist, nebulae, ointment, cream, gel, paste, salve, solution, suspension, tincture, patch, and atomized vapor.

6. The nasal formulation of claim 2, wherein said natural antifungal agent is chosen from oregano oil, berberine, garlic oil, allicin, alliin, alliinase, cellulase, S-allylcysteine, colloidal silver, tannin, Lactobacillus acidophilus, Lactobacillus rhamnosus, Lactobacillus bulgaricus, Lactobacillus salivarius, Lactobacillus plantarum, Lactobacillus casei, and Lactobacillus sporogenes, Bifidobacteria bifidum, Bifidobacteria longum, and Bifidobacteria infantis.

7. The nasal formulation of claim 6, wherein said formulation is in a solid, liquid or aerosol form.

8. The nasal formulation of claim 6, wherein said formulation is in a form chosen from a spray, aerosol, powder, mist, nebulae, ointment, cream, gel, paste, salve, solution, suspension, tincture, patch, and atomized vapor.

9. A nasal formulation for improving nasal airflow of a human suffering from nasal congestion, comprising a synergistic amount of a pharmaceutical antifungal agent or a combination thereof in combination with a compound chosen from a natural antifungal agent, a vasoconstrictor, a glucocorticosteroid, an antihistamine, an anticholinergic, a mucolytic, and a combination thereof, in association with a nasal inert carrier.

10. The nasal formulation of claim 9, wherein said pharmaceutical antifungal agent is chosen from terbinafine, butenafine, naftifine, butoconazole, clotrimazole, econazole, fluconazole, itraconazole, ketoconazole, miconazole, oxiconazole, sulconazole, sertaconazole, terconazole, saperconazole, tinidazole, tioconazole, voriconazole, nyastatin, micafungin, griseofulvin, haloprogin, mafenide, mycafungin, caspofungin, flucytosine, natamycin, undecylenic acid, caprylic acid, propionic acid, ciclopyrox olamine, amphotericin B, and tolnaftate.

11. The nasal formulation of claim 10, wherein said natural antifungal agent is chosen from oregano oil, berberine, garlic oil, allicin, alliin, alliinase, cellulase, S-allylcysteine, colloidal silver, tannin, Lactobacillus acidophilus, Lactobacillus rhamnosus, Lactobacillus bulgaricus, Lactobacillus salivarius, Lactobacillus plantarum, Lactobacillus casei, and Lactobacillus sporogenes, Bifidobacteria bifidum, Bifidobacteria longum, and Bifidobacteria infantis.

12. The nasal formulation of claim 11, wherein said formulation is in a solid, liquid or aerosol form.

13. The nasal formulation of claim 11, wherein said formulation is in a form chosen from a spray, aerosol, powder, mist, nebulae, ointment, cream, gel, paste, salve, solution, suspension, tincture, patch, and atomized vapor.

14. A method for improving nasal airflow of a human suffering from nasal congestion, said method comprising administering nasally to said human an effective amount of a nasal formulation according to claim 1.

15. A method for improving nasal airflow of a human suffering from nasal congestion, said method comprising administering nasally to said human an effective amount of a nasal formulation according to claim 13.
